(19) United States
(12) Patent Application Publication
(10) Pub. No.: US 2023/0009158 A1
(43) Pub. Date: Jan. 12, 2023

(54) BAND FOR WEARABLE NEUROSTIMULATION SYSTEM

(71) Applicant: Cala Health, Inc., San Mateo, CA (US)

(72) Inventor: Jessica M. Liberatore, San Mateo, CA (US)

(21) Appl. No.: 17/808,850

(22) Filed: Jun. 24, 2022

Related U.S. Application Data
(60) Provisional application No. 63/203,150, filed on Jul. 9, 2021.

(51) Int. Cl. A61N 1/04; A61N 1/36

(52) U.S. Cl. CPC A61N 1/0484; A61N 1/0456; A61N 1/36014

(57) ABSTRACT

A band to be worn by a user and for securing an enclosure of a wearable neurostimulation device that stimulates one or more peripheral nerves of a user is provided. The band may include a flexible platform that bends from a first shape to a second shape. The band may include a plurality of lugs that have an engagement structure configured to mechanically engage with the enclosure when the flexible platform is in the first shape and disengage from the enclosure when the flexible platform is in the second shape. The band can further include an electrode system that has an inner side and an outer side. The inner side having at least one electrode for each nerve to be stimulated.

### CROSS-REFERENCE TO RELATED APPLICATION

This application claims benefit under 35 U.S.C. 119(e) to U.S. Provisional Patent App. No. 63/203,150, filed Jul. 9, 2021, the entire disclosure of which is hereby incorporated by reference herein in its entirety. Any and all priority claims identified in the Application Data Sheet, or any corrections thereto, are hereby incorporated by reference under 37 CFR 1.57.

### FIELD

Some embodiments of the invention relate generally to systems, devices, methods for neuromodulating (such as stimulating) nerves, and methods of manufacture. Several embodiments include, for example, bands for releasably securing a wearable device on a user's limb for electrically stimulating peripheral nerve(s) to treat various diseases and disorders.

### DESCRIPTION OF THE RELATED ART

A wide variety of modalities can be utilized to neuromodulate peripheral nerves. For example, Applicant's own work has demonstrated that electrical energy can be delivered transcutaneously via electrodes on the skin surface with neurostimulation systems to stimulate peripheral nerves, including on a patient's limb.

### SUMMARY

Wearable systems to neuromodulate nerves with compact, ergonomic form factors are needed to enhance efficacy, compliance, and/or comfort with using the devices. The devices can be attached to a band that is wrapped around a patient's wrist. The band is worn throughout the day including during daily activities. To provide the ability for the patient to change or replace the band while keeping the same device, the device is not permanently coupled to the band. This feature allows the patient to swap out or upgrade their band without incurring the cost of purchasing a new device. This feature further allows the patient to switch their device between bands that are designed for different purposes and/or activities. Characteristics of each band (e.g., material, weight, size, color, etc.) can be optimized or selected depending on the purpose or activity. The patient may simply disengage their device from their current band and then engage the same device on another band that has been optimized for the upcoming activity.

In some embodiments, because the device and band are used during daily activities and are not permanently coupled together, the engagement between the band and the device needs to be secure while also being ergonomic in allowing the patient to easily remove the device from the band without relying on hand tools (e.g., screwdriver, wrench, pliers). Simple attachment structures such as snaps may not provide the desired level of securement between the device and the band.

Some embodiments of the system disclosed herein improve the level or degree of attachment between the device and the band by requiring the patient to use different motions to attach and detach the device from the band. In some embodiments, the patient removes the device from the band by pinching lugs of the band towards each to cause the band to flex or bend. When in the flexed or bent state, the device can be lifted off the band. In some embodiments, the bottom of the band forms a convex shape when in the flexed or bent state. In some embodiments, the patient may find it easier to bend or flex the band to the convex shape after they have removed the band from their wrist. Typically, when worn, the band is tightly located against the patient's wrist which prevents the band from flexing enough to inadvertently release the device. In some embodiments, magnets are employed to solely attach the device to the band as well can be employed in combination with other structures (e.g., hooks, tangs, lips, slots, keyways, etc.) for attaching the device to the band.

In some embodiments, disclosed herein is a band for securing an enclosure of a wearable neurostimulation device for modulating one or more peripheral nerves of a user. In some embodiments, the band includes a flexible platform configured to bend from a first shape to a second shape different than the first shape responsive to an applied force, a plurality of lugs supported by the flexible platform, the plurality of lugs having engagement structures configured to mechanically engage with the enclosure of the wearable neurostimulation device when the flexible platform is in the first shape and disengage from the enclosure when the flexible platform is moved to the second shape responsive to the applied force, and an electrode system coupled to the flexible platform and having an inner side and an outer side, the inner side comprising at least one electrode for each nerve to be stimulated.

In some embodiments, the first shape is generally planar and the second shape is curved.

In some embodiments, the engagement structure comprises on or more hooks.

In some embodiments, the band further includes one or more magnets, the one or more magnets being configured to create a magnetic attractive force with the enclosure when the enclosure is in close proximity to the band.

In some embodiments, the flexible platform comprises a groove configured to reduce a bending force required to bend the flexible platform from the first shape to the second shape.

In some embodiments, at least one of the plurality of lugs comprises one or more holes for providing a passage for an electrical contact from the enclosure to contact the electrode system.

In some embodiments, the electrode system comprises a distal end and a proximal end, and wherein the electrical contact from the enclosure is configured to contact the distal end.

In some embodiments, the band further includes a strap, wherein the plurality of lugs further comprise a plurality of apertures configured to receive portions of the strap at least when the strap encircles a limb of the user.

In some embodiments, the band further comprises the wearable neurostimulation device, wherein the enclosure forms a portion of the wearable neurostimulation device.

In some embodiments, disclosed herein is a band for securing an enclosure of a wearable neurostimulation device that stimulates one or more peripheral nerves of a user. In some embodiment, the band includes a platform configured to bend from a first shape to a second shape different than the first shape, an engagement structure configured to secure the enclosure to the platform when the platform is in the first shape and disengage from the enclosure when the platform is in the second shape, and a plurality of contact surfaces configure for the user to bend the platform from the first shape to the second shape.

In some embodiments, the band further comprises an electrode system coupled to the flexible platform and having an inner side and an outer side, the inner side comprising at least one electrode for each nerve to be stimulated.

In some embodiments, the first shape is generally planar and the second shape is curved.

In some embodiments, the engagement structure comprises on or more hooks.

In some embodiments, the band further includes one or more magnets, the one or more magnets being configured to create a magnetic attractive force with the enclosure when the enclosure is in close proximity to the band.

In some embodiments, the platform is flexible. In one embodiment, varying regions of different flexibility/rigidity are provided.

In some embodiments, the platform comprises a groove configured to reduce a bending force required to bend the platform from the first shape to the second shape.

In some embodiments, the band further includes a plurality of lugs supported by the platform, the engagement structure and the plurality of contact surfaces being disposed on the plurality of lugs.

In some embodiments, the band further includes a strap, at least one end of the strap being fixed to the platform.

In some embodiments, the at least one electrode comprises at least a first electrode and a second electrode, the first electrode being configured to stimulate the median nerve of the user and the second electrode being configured to stimulate the radial or ulnar nerve of the user. In one embodiment, a first electrode stimulates the median nerve, a second electrode stimulates the radial nerve, and a third electrode stimulates the ulnar nerve. In one embodiment, two or more electrodes stimulate the same nerve (e.g., with different frequencies or other parameters). In one embodiment, one two or all of the median nerve, radial nerve, and ulnar nerve are stimulated.

In some embodiments, the at least one electrode comprises a return or ground electrode configured to be electrically coupled to the user.

In some embodiments, the band further includes the wearable neurostimulation device, wherein the enclosure forms a portion of the wearable neurostimulation device.

In some embodiments, disclosed herein is a method of manufacturing a band for securing an enclosure of a wearable neurostimulation device that stimulates one or more peripheral nerves of a user. In some embodiments, the method includes over molding one or more electrodes and a conductive material to form an internal layer, the one or more electrodes being exposed within the conductive material and over molding the internal layer with a non-conductive outer layer to form an electrode system, the one or more electrodes being exposed within the non-conductive material.

In some embodiments, the method further includes providing a flexible platform configured to bend from a first shape to a second shape different than the first shape.

In some embodiments, the method further includes providing a plurality of lugs having an engagement structure configured to mechanically engage with the enclosure when the flexible platform is in the first shape and disengage from the enclosure when the flexible platform is in the second shape.

In some embodiments, the method further includes over molding a proximal end of the electrode system, the plurality of lugs, and the flexible platform to form the band.

In some embodiments, disclosed herein is a band for securing an enclosure of a wearable neurostimulation device that stimulates one or more peripheral nerves of a user. In some embodiments, the band includes a platform configured to support the wearable neurostimulation device, an electrode system coupled to the platform and having an inner side and an outer side, the inner side comprising at least one electrode for each nerve to be stimulated, and a strap coupled to the platform and being sized and shaped to wrap around the outer side of the electrode system when encircling a limb of the user.

In some embodiments, the electrode system comprises a distal end and a proximal end, and wherein only the proximal end is coupled to the platform.

In some embodiments, the electrode system comprises one or more electrical traces extending between the proximal end and the at least one electrode.

In some embodiments, the coupling between the electrode system and the platform includes a mechanical coupling and an electrical coupling.

In some embodiments, the platform is flexible. In one embodiment, varying regions of different flexibility/rigidity are provided.

In some embodiments, the strap is configured to be tightened against the outer surface of the electrode system forcing the at least one electrode firmly against skin of the user.

In some embodiments, the distal end of the strap comprises an aperture sized and shaped to slidingly receive the strap.

In some embodiments, the aperture is configured to inhibit the distal end of the electrode system from sliding along the limb of the user when both ends of the strap are secured relative to the platform.

In some embodiments, disclosed herein is a strap configured to secure a neurostimulation system on a wrist of a user. In some embodiments the strap includes a first portion adjacent to a first end along a length of the strap, said first portion configured to be secured to a first position of a platform of the neurostimulation system, a second portion along the length of the strap, said second portion configured to be secured to an electrode system coupled to the platform, a third portion along the length of the strap, said third portion configured to be secured to a second position of the platform, said second position substantially at an opposite end of the first position, and a fourth portion adjacent to a second end along the length of the strap, said fourth portion comprising an attachment mechanism configured to secure the fourth portion to an outer surface of the strap, said outer surface being opposite to an inner surface of the strap, the inner surface being configured to be in contact with the wrist of the user.

In some embodiments, the first portion forms a loop when secured to the first position of the platform.

In some embodiments, the second portion is secured to the electrode system by passing through an aperture in the electrode system.

In some embodiments, the third portion forms a loop when secured to the second position of the platform.

In some embodiments, the fourth portion comprises hook and loop fasteners.

In some embodiments, the strap has an adjustable length.

In some embodiments, the strap is sized for various sizes of users.

In some embodiments, the strap is offset toward or near one side of the length of the platform.

In some embodiments, the strap has a width that is less than a maximum width of the electrode system.

In some embodiments, disclosed herein is a wrist wearable system configured to removably secure a controller. In some embodiments, the wrist wearable system can include a platform comprises an engagement system configured to engage the controller and a strap extending from a first portion of the platform, wherein an end of the strap is not secured to the platform.

In some embodiments, the platform is configured to bend from a first shape to a second shape different than the first shape to allow the controller to be disengaged from the platform.

In some embodiments, disclosed herein is a method for securing an enclosure of a wearable device. In some embodiments, the method can include providing a flexible platform that bends from a first shape to a second shape different than the first shape responsive to an applied force, mechanically engaging the enclosure of the wearable device with the flexible platform when the flexible platform is in the first shape, and disengaging the enclosure from the flexible platform when the flexible platform is moved to the second shape responsive to the applied force.

In some embodiments, disclosed herein is a method for releasably securing an enclosure of a wearable device to a flexible platform of a band. In some embodiments, the method can include mechanically engaging the enclosure with the flexible platform, bending the flexible platform from a first shape when the flexible platform is mechanically engaged with the flexible platform to a second shape different than the first shape, and disengaging the enclosure from the flexible platform when the flexible platform is in the second shape.

In some embodiments, the method further can include aligning the enclosure above the flexible platform and lowering the enclosure towards the flexible platform.

In some embodiments, mechanically engaging the enclosure comprises deflecting one or more hooks extending from the flexible platform.

In some embodiments, mechanically engaging the enclosure comprises creating magnetic attraction between the flexible platform and the enclosure.

In some embodiments, mechanically engaging the enclosure comprises pressing the enclosure against a surface of the flexible platform.

In some embodiments, pressing the enclosure against the surface of the flexible platform is performed when the flexible platform is in the first shape.

In some embodiments, pressing the enclosure against the surface of the flexible platform comprises at least one of deflecting one or hooks, creating magnetic attraction between the enclosure and the flexible platform, or bending the flexible platform from the first shape slightly towards the second shape.

In some embodiments, bending the flexible platform comprises pinching lugs coupled to the flexible platform towards the enclosure.

In some embodiments, the lugs are pinched in a direction towards each other.

In some embodiments, bending the flexible platform comprises deflecting one or more hooks.

In some embodiments, the one or more hooks are deflected in a direction towards each other.

In some embodiments, a first portion of the flexible platform is spaced further from the enclosure than a second portion of the flexible platform when the flexible platform is bent to the second shape.

In some embodiments, the first portion is a center of the flexible platform.

In some embodiments, bending the flexible platform comprises forming a gap between the flexible platform and the enclosure.

In some embodiments, the gap is U-shaped.

In some embodiments, the gap is V-shaped.

In some embodiments, the first shape is generally planar and the second shape is generally curved.

In some embodiments, disengaging the enclosure from the flexible platform comprises lifting the enclosure away from the flexible platform.

In some embodiments, disengaging the enclosure from the flexible platform comprises overcoming magnetic attraction between the enclosure and the flexible platform.

In some embodiments, bending the flexible platform and disengaging the enclosure overlap at least partially in time.

In some embodiments, the method further includes wrapping the band around a wrist of a user.

In some embodiments, wrapping the band around the wrist comprises contacting the wrist with an electrode system.

In some embodiments, the method further includes electrically stimulating one or more nerves via the electrode system.

In some embodiments, wrapping the band around the wrist comprises wrapping a strap around an outer surface of the electrode system.

In some embodiments, the method further includes electrically engaging the enclosure with the band when the flexible platform is in the first shape.

In some embodiments, the method further includes electrically stimulating one or more nerves via a plurality of electrodes.

In some embodiments, electrically stimulating one or more nerves via the plurality of electrodes comprises conducting a stimulation signal between the enclosure and the flexible platform.

In some embodiments, any of the devices or methods are used for treatment of depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease).

In some embodiments, the band is used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases.

In some embodiments, the band is used for the treatment of inflammatory skin conditions.

In some embodiments, the band is used for the treatment of chronic fatigue syndrome.

In some embodiments, the band is used for the treatment of chronic inflammatory symptoms and flare ups.

In some embodiments, the band is used for the treatment of cardiac conditions (such as atrial fibrillation, hypertension, epilepsy, and stroke).

In some embodiments, the band is used for the treatment of immune dysfunction.

In some embodiments, the band is used to stimulate the autonomic nervous system.

In some embodiments, the band is used to balance the sympathetic/parasympathetic nervous systems.

In some embodiments, the band is used in a system and/or method which further comprises a wrist worn device (e.g., that fully or partially encircles the wrist).

In some embodiments, the devices or methods are used for treatment of disorders and/or associated symptoms such as depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). In some embodiments, conditions such as stroke, cardiac events, inflammation, etc. are treated.

In several embodiments, one or more of bradykinesia, dyskinesia, gait dysfunction, dystonia and/or rigidity are treated with the devices and methods described herein (e.g., in connection with Parkinson's disease or in connection with other disorders). Rehabilitation of movement is treated in some embodiments (for example to restore or improve movement and motion) in subjects who have suffered from an acute or chronic event, including, for example, cardiac events (such as atrial fibrillation, hypertension, epilepsy, and stroke), inflammation, neuroinflammation, etc.). Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One, two, three or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection are treated, including for example, inflammation (e.g., neuroinflammation), headache, back pain, joint pain and stiffness, muscle pain and tension, etc.

### BRIEF DESCRIPTION OF THE DRAWINGS

The following drawings are for illustrative purposes only and show non-limiting embodiments. Features from different figures may be combined in several embodiments.

FIG. 1 is a perspective view of an embodiment of a band configured to be worn by a user and to detachably couple to an enclosure of a device for providing transcutaneous peripheral nerve stimulation to the user.

FIG. 2A is a bottom side view of a platform and an electrode system from FIG. 1 coupled to the enclosure and showing a groove in the platform which reduces the bending force applied by the user to remove the enclosure from the band. FIG. 2B is a side view taken along lines 2B-2B in FIG. 2A. FIG. 2C is an end view taken along lines 2C-2C in FIG. 2A. FIGS. 2A-2C shows one design embodiment of the size, shape, number and placement of electrodes. In other embodiments, 3-12 electrodes are used, where the electrodes may be oval, circular or square in shape, as an example.

FIG. 3 is a perspective view of the band from FIG. 1 with one or more magnets of the enclosure aligned with one or more magnets of the band prior to attachment of the enclosure to the platform in part by an attractive magnetic force.

FIG. 4 is a perspective view similar to FIG. 3 except the enclosure of the device is attached to the platform of the band.

FIG. 5 is a front view of the enclosure and band from FIG. 4 showing opposing lugs that are configured to be pinched by the user to facilitate removal of the enclosure from the platform of the band.

FIG. 6A is a top plan view of an internal layer of an electrode system that includes one or more electrodes and a conductive material. The one or more electrodes are configured to contact skin of the user.

FIG. 6B is a view similar to FIG. 6A except portions of the internal layer are covered by an outer layer of non-conductive material leaving the one or more electrodes uncovered. FIGS. 6A-6B shows one design embodiment of the size, shape, number and placement of electrodes. In other embodiments, 3-12 electrodes are used, where the electrodes may be oval, circular or square in shape, as an example.

FIG. 6C is a bottom plan view of the electrode system from FIG. 6B.

FIG. 7 is perspective views of a first lug and a second lug from FIG. 1.

FIG. 8 is plan views of the first lug and the second lug from FIG. 7.

FIG. 9 is a perspective view of the electrode system from FIGS. 6B-6C assembled to the platform by sandwiching a proximal end of the electrode system between the first lug and the platform. The electrode system is disposed so as to contact one or more electrical contacts of the enclosure when the enclosure is secured to the platform.

FIG. 10 is similar to FIG. 9 except one or more magnets are disposed in each of the first and second lugs to create a magnetic attraction force between the enclosure and the platform.

### DETAILED DESCRIPTION

Disclosed herein are bands for securing an enclosure of a device configured for providing neuromodulation (e.g., neurostimulation). The band provided herein can be configured to attach to an enclosure of a device that stimulates peripheral nerves of a user. The enclosure of the device may be configured to be coupled to the surface of a user's skin for transcutaneous stimulation using the band.

The devices provided herein may be configured to stimulate peripheral nerves of a user. The neuromodulation (e.g., neurostimulation) devices may be configured to transmit one or more neuromodulation (e.g., neurostimulation) signals across the skin of the user. In many embodiments, the devices are wearable devices configured to be worn by a user. The user may be a human, another mammal, or other animal user. The system could also include signal processing systems and methods for enhancing diagnostic and therapeutic protocols relating to the same. In some embodiments, the device is configured to be wearable on an upper extremity of a user (e.g., a wrist, forearm, arm, and/or finger(s) of a user). In some embodiments, the device is configured to be wearable on a lower extremity (e.g., ankle, calf, knee, thigh, foot, and/or toes) of a user. In some embodiments, the device is configured to be wearable on the head or neck (e.g., forehead, ear, neck, nose, and/or tongue). In several embodiments, dampening or blocking of nerve impulses and/or neurotransmitters are provided. In some embodiments, nerve impulses and/or neurotransmitters are enhanced. In some embodiments, the device is configured to be wearable on or proximate an ear of a user, including but not limited to auricular neuromodulation (e.g., neurostimulation) of the auricular branch of the vagus nerve, for example. Clips, bands, ear buds, etc. may be used for auricular stimulation, and may be used for example, together with a band for the arm or leg. In some embodiments, the vagus nerve, trigeminal nerve and/or greater auricular nerve is/are neuromodulated. In some embodiments, only the vagus nerve is neuromodulated. In some embodiments, the vagus nerve and one, two or more other nerves are neuromodulated (e.g., trigeminal nerve, greater auricular nerve, nerves of the auricular branch, auricular branch of the vagus nerve, the facial nerve, the auriculotemporal nerve etc.). In some embodiments, the vagus nerve is not stimulated and instead, for example, another nerve is stimulated (e.g., trigeminal nerve, greater auricular nerve, the facial nerve, the auriculotemporal nerve, other nerves of the auricular branch, etc.). One or more of the vagus, tragus, trigeminal or cranial nerves may be treated in some embodiments. The device could be unilateral or bilateral, including a single device or multiple devices connected with wires or wirelessly. Transcutaneous neuromodulation is provided in several embodiments, although subcutaneous and percutaneous components may also be used. In some embodiments, the device includes three to six or more electrodes (e.g., 3, 4, 5, 6), and is partially implantable or is entirely transcutaneous.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, greater auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

Single or multiple bands that partially or fully encircle a limb (such as a wrist, ankle, arm, leg) are provided in some embodiments. Ear devices are also provided in some embodiments that can be used with or without a limb band. In one embodiment, an ear device and a wrist band are provided for synergistic treatment. An auricular (e.g., ear) device can include an earpiece or bud for one or more portions of the ear such as an ear canal or external ear. One to six or more electrodes may be placed on the earpiece or bud, or on a device connected to the earpiece/bud. Right, left or two earpieces are provided in some embodiments.

Systems with compact, ergonomic form factors are needed to enhance efficacy, compliance, and/or comfort when using non-invasive or wearable neuromodulation devices. In several embodiments, neuromodulation systems and methods are provided that enhance or inhibit nerve impulses and/or neurotransmission, and/or modulate excitability of nerves, neurons, neural circuitry, and/or other neuroanatomy that affects activation of nerves and/or neurons. For example, neuromodulation (e.g., neurostimulation) can include one or more of the following effects on neural tissue: depolarizing the neurons such that the neurons fire action potentials; hyperpolarizing the neurons to inhibit action potentials; depleting neuron ion stores to inhibit firing action potentials; altering with proprioceptive input; influencing muscle contractions; affecting changes in neurotransmitter release or uptake; and/or inhibiting firing.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, low frequency stimulation (e.g., less than about 5 Hz, 100 Hz, 150 Hz, 200 Hz, in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

Stimulation of peripheral nerves can provide therapeutic benefit across a variety of diseases, including but not limited to disorders and/or associated symptoms such as movement disorders (including but not limited to essential tremor, Parkinson's tremor, orthostatic tremor, and multiple sclerosis), urological disorders, gastrointestinal disorders, cardiac diseases, inflammatory diseases, mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, epilepsy, and stroke) are treated in one embodiment. Inflammatory skin conditions and immune dysfunction are also treated in some embodiments. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s).

In several embodiments, one or more of bradykinesia, dyskinesia, gait dysfunction, dystonia and/or rigidity are treated. These may be treated in connection with Parkinson's disease or in connection with other disorders. Rehabilitation of movement is treated in some embodiments (for example to restore or improve movement and motion) in subjects who have suffered from an acute or chronic event including, for example, cardiac events (such as atrial fibrillation, hypertension, epilepsy, and stroke), inflammation, neuroinflammation, etc.). Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation), headache, back pain, joint pain and stiffness, muscle pain and tension, etc.

In some embodiments, wearable systems and methods as disclosed herein can advantageously be used to identify whether a treatment is effective in significantly reducing or preventing a medical condition, including but not limited to tremor severity. Although tremor is treated in several embodiments, the devices described herein are used to treat conditions other than tremor. Wearable sensors can advantageously monitor, characterize, and aid in the clinical management of hand tremor as well as other medical conditions including those disclosed elsewhere herein. Not to be limited by theory, clinical ratings of medical conditions, e.g., tremor severity can correlate with simultaneous measurements of wrist motion using inertial measurement units (IMUs). For example, tremor features extracted from IMUs at the wrist can provide characteristic information about tremor phenotypes that may be leveraged to improve diagnosis, prognosis, and/or therapeutic outcomes. Kinematic measures can correlate with tremor severity, and machine learning algorithms incorporated in neuromodulation systems and methods as disclosed for example herein can predict tremor severity.

In other non-tremor embodiments, physiological data including heart rate, blood glucose, blood pressure, respiration rate, body temperature, blood volume, sound pressure, photoplethysmography, electroencephalogram, electrocardiogram, blood oxygen saturation, and/or skin conductance as well as patient data from third party devices can be collected and/or aggregated to improve diagnosis, prognosis, and/or therapeutic outcomes for migraine, depression, and/or Lyme disease. For example, physiological data including respiration rate and heart rate along with data related to sleep patterns and activity level can be collected and/or aggregated to improve diagnosis, prognosis, and/or therapeutic outcomes for depression.

In several embodiments, neuromodulation, such as neurostimulation, as used herein is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, GI issues, nausea, confusion, dyskinesia, altered appetite, etc.

In several embodiments, a device (such as a band, strap, bracelet, cuff or other device that partially or fully encircles a limb) is provided for securing an enclosure of a wearable neurostimulation device for modulating one or more peripheral nerves of a user, the device comprising a flexible platform configured to bend from a first shape to a second shape different than the first shape responsive to an applied force. Also included in one embodiment is a plurality of lugs supported by the flexible platform, the plurality of lugs having engagement structures configured to mechanically engage with the enclosure of the wearable neurostimulation device when the flexible platform is in the first shape and disengage from the enclosure when the flexible platform is moved to the second shape responsive to the applied force. The device may also include an electrode system coupled to the flexible platform and having an inner side and an outer side, the inner side comprising at least one electrode (1, 2, 3, 4, 5, 6, 7, 8 or more) for each nerve to be stimulated. The first shape may be generally planar and/or the second shape may be curved. The engagement structure may comprise one or more hooks. The device optionally includes one or more magnets, the one or more magnets being configured to create a magnetic attractive force with the enclosure when the enclosure is in close proximity to the device. The flexible platform comprises, in one embodiment, a groove configured to reduce a bending force required to bend the flexible platform from the first shape to the second shape. At least one of the plurality of lugs may comprise one or more holes for providing a passage for an electrical contact from the enclosure to contact the electrode system. The electrode system comprises, for example, a distal end and a proximal end, and wherein the electrical contact from the enclosure is configured to contact the distal end. The electrode system can comprise one or more electrical traces extending between the proximal end and the at least one electrode. The coupling between the electrode system and the platform can include a mechanical coupling and an electrical coupling.

In some embodiments, the band allows the user to attach the enclosure to the band with one motion and remove the enclosure with another motion. For example, in some embodiments, to attach the enclosure, magnets in the band and the enclosure align themselves and pull the platform against the bottom of the enclosure. In some embodiments, hooks engage with the enclosure. To remove the enclosure, the user pinches lugs of the band toward the enclosure which allows the platform of the band to flex disengaging the hooks from the enclosure. While being worn in some embodiments, the platform is tightly located against the wrist of the user which reduces the degree of flex exhibited by the platform so that the user is unable to remove the enclosure even if the user pinches the lugs together. In this way, by inhibiting the platform from bending or flexing when being worn, it is less likely that the enclosure could inadvertently disengage from the platform.

Disclosure herein are methods for manufacturing the band in some embodiments. In some embodiments, a method includes over molding one or more electrodes with a conductive silicone leaving a surface of the one or more electrodes uncovered. In some embodiments, a non-conductive silicone is then over molded over the conductive silicone leaving the one or more electrodes as well as a proximal end of the electrode system uncovered. A first lug and a second lug are molded with each including one or more engagement structures configured to secure to the enclosure. In some embodiments, the first and second lugs each include a hook and magnet for securing the enclosure to the lugs. In some embodiments, the platform is over molded with the first and second lugs while the proximal end of the electrode system is sandwiched between the first leg and the platform. In some embodiments, a strap attaches to the first lug, passes through an aperture in the distal end of the electrode system and then loops through the second lug to allow the user to press the electrode system tightly against the skin by tightening the strap.

In several embodiments, neuromodulation, such as neurostimulation, as used herein is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, GI issues, nausea, confusion, dyskinesia, altered appetite, etc.

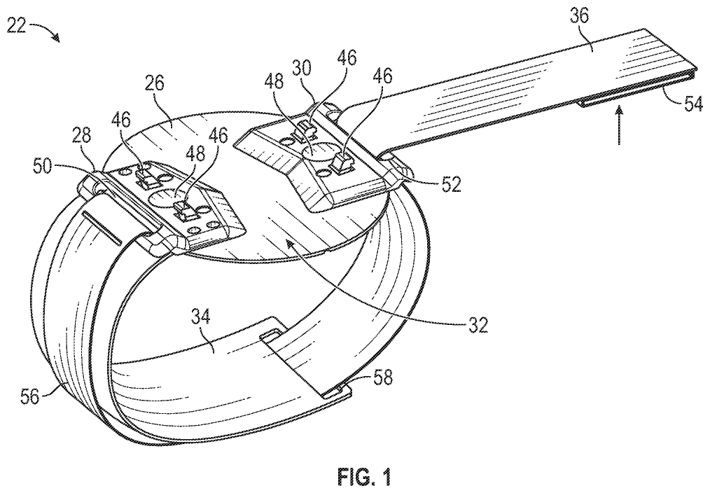

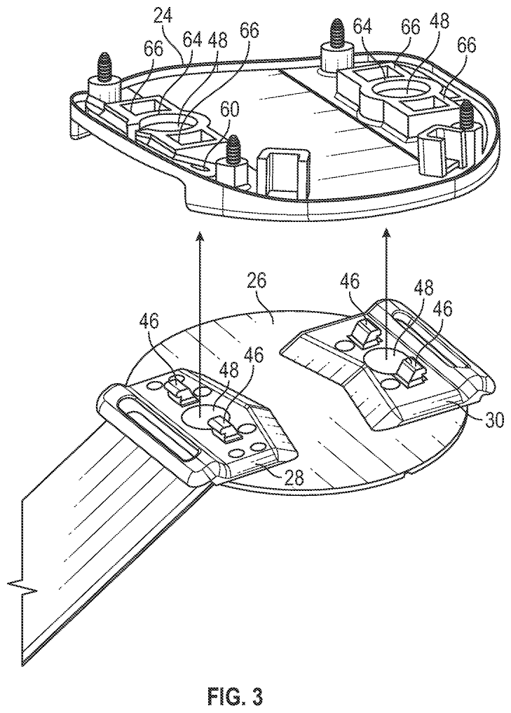

FIG. 1 is a perspective view of an embodiment of a band 22 configured to be worn by a user and to detachably couple to a bottom surface of an enclosure 24 of a device 20 for providing transcutaneous peripheral nerve stimulation to the user. In some embodiments, the band 22 is configured to be mechanically and electrically coupled to the enclosure 24 (see FIG. 3). In many embodiments, the transcutaneous device 20 is a wearable band or earpiece. The band 22 may partially or fully surround a wrist, finger, arm, leg, ankle or head. Patches may be used, but in many embodiments a patch is not used.

The enclosure 24 may have a top surface and an opposing bottom surface. Only the bottom surface of the enclosure 24 is shown in FIG. 3. The enclosure 24 may have a sidewall extending from the top surface to the bottom surface and defining a height of the enclosure 24. The top surface and the bottom surface may have substantially the same shape. For example, the top surface and the bottom surface may be substantially rectangular, substantially oval, or an intermediate shape between a rectangle and an oval. In other embodiments, the shape may be circular, triangular, polygonal, etc.

The entire enclosure 24 may be configured to enclose or contain electronic circuitry for generating and providing a neurostimulation signal to be applied to the user. The circuitry may be self-contained in the enclosure 24 such that the neurostimulation device 20 is portable. The circuitry may include a pulse generator for generating an electrical stimulation pulse and a controller for controlling the delivery of the electrical pulses. The enclosure 24 may also comprise a power source, such as a battery. The enclosure 24 may also contain one or more processors and memory. The enclosure 24 may comprise one or more displays (e.g., digital displays, LEDs, etc.) to display information to the user, such as on a top surface of the enclosure 24. Displays may also be touch-sensitive to receive inputs from the user. The enclosure 24 may comprise one or more audio signal generators. The enclosure 24 may comprise antennas for wireless communication. The enclosure 24 may also comprise a haptic motor to provide feedback or notification to the wearer by vibration. The enclosure 24 may comprise one or more interface features, such as depressible or solid-state buttons for example, by which a user may interface with the device 20.

The band 22 may be configured to be worn by a user around his or her arm, wrist finger, leg, ankle, knee, waist, etc. In some embodiments, the band 22 comprises an electrode system 34 for distributing electrical stimulation signals generated by the enclosure 24 to the skin of the user.

The band 22 may be configured to hold the enclosure 24 close to the user. In some embodiments, the closure of the band 22 may be configured as a D-shaped loop as shown in FIG. 1. In the illustrated embodiment, the band 22 includes a strap 36 which secures and tightens the band 22, including the electrode system 34, to the user. In some embodiments, the band 22 is configured with a clasp or buckle that secures and tightens the band 22 on the wrist of the user.

The band 22 may comprise a platform 26 and one or more lugs 28, 30 In some embodiments. In the illustrated embodiment, the band 22 comprises a first lug 28 and a second lug 30 supported by the platform 26. In some embodiments, the platform 26 and the one or more lugs 28, 30 are manufactured as a unitary structure. In some embodiments, the platform 26 and the one or more lugs 28, 30 are shaped and sized to sit at least partially underneath the enclosure 24.

In some embodiments, the first and second lugs 28, 30 are configured to mechanically and/or electrically interface with the enclosure 24. In some embodiments, the first and second lugs 28, 30 couple to the platform 26 of the band 22 and extend upward from a top surface of the platform 26. The first and second lugs 28, 30 may substantially mirror a portion of a bottom surface of the enclosure 24 comprising electrical contacts 60, as seen in FIG. 3. In the illustrated embodiment, the electrical contacts 60 can be located on a bottom side or surface of the enclosure 24.

In some embodiments, the enclosure 24 and the first and second lugs 28, 30 may comprise corresponding keying features which ensure the enclosure 24 and the band 22 are coupled in an appropriate orientation. In the illustrated embodiment, the first lug 28 and the second lug 30 are spaced from each other on the platform 26 and form a channel 32 therebetween. In the illustrated embodiment, the channel 32 has a V-shape. Of course, the channel 32 need not have a V-shape and can have any other shape. In the illustrated embodiment, the channel 32 has an asymmetric shape across an axis passing between the first and seconds lugs 28, 30. In some embodiments where a bottom of the enclosure 24 has a shape that matches the shape of the channel 32, the asymmetric shape requires the user to attach the enclosure 24 in a single orientation. The keying features may ensure, for example, that the electrical contacts 60 are connected through the proper one or more holes 44 and not reversed. The keying features may be particularly advantageous for embodiments where the electrical contacts 60 form a symmetric arrangement. The keying features may ensure that the proper stimulation signal is electrically coupled to the proper electrode 38, 40 of the electrode system 34 and, correspondingly, the proper nerve, and prevent the device 20 from being worn on the wrong hand (e.g., right or left hand).

The asymmetric shape of the channel 32, in addition to the engagement features describe below, may further prevent relative rotation of the enclosure 24 when secured to the band 22. In this way, the enclosure 24 may be configured (e.g., shaped and sized) to be received in the channel 32 of the band 22. For example, the first and second lugs 28, 30 may have any appropriate shape including those described elsewhere herein and a general height matched to the depth of recesses in the bottom surface of the enclosure 24.

The first and second lugs 28, 30 may form a reversibly detachable interference fit or snap fit with the enclosure 24. In some embodiments, the first and second lugs 28, 30 may comprise a recess and the enclosure 24 may comprise a projection. Positioning the electrical stimulation contacts 60 within the first lug 28 or the second lug 30 may advantageously protect the electrical stimulation contacts 60 from damage.

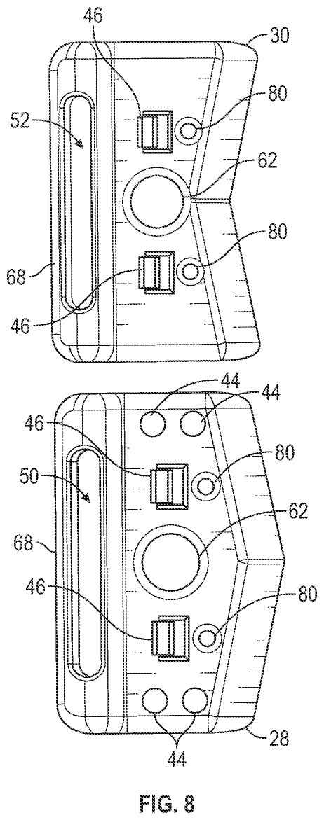

In some embodiments, the one or more lugs 28, 30 may encompass all the electrical contacts with the enclosure 24. For example, in the illustrated embodiment, one or more holes 44 in the first lug 28 provide passages for the electrical contacts 60 (e.g., ground and stimulation) (see FIG. 3) from the enclosure 24 to contact the electrode system 34. The holes may be round, as shown in FIG. 8, oval, elliptical/stadium shaped, or any other suitable shape. The electrical contacts 60 from the enclosure 24 may be snap connections which form snap fits (e.g., annular snap fits) with corresponding contacts or holes on the first lug 28 and/or the electrode system 34.

The electrical contacts 60 may deliver or transfer electrical signals to the electrode system 34. The electrical contacts 60 may be positioned on the bottom surface of the enclosure 24. The electrical contacts 60 may include one electrical stimulation contact for each electrode 38, 40 to be applied to the user. The electrical contacts 60 may include at least one electrical stimulation contact for each nerve that is to be stimulated. For example, the electrical contacts 60 may include an electrical stimulation contact configured to deliver a signal to the median nerve, the radial nerve, the ulnar nerve or any combination thereof. In some embodiments, stimulation may alternate between each nerve such that the nerves are not stimulated simultaneously. In some embodiments, all nerves are stimulated simultaneously. In some embodiments, stimulation is delivered to the various nerves in one of many bursting patterns. The stimulation parameters may include on/off, time duration, intensity, pulse rate, pulse width, waveform shape, and the ramp of pulse on and off. In one embodiment the pulse rate may be from about 1 to about 5000 Hz, about 1 Hz to about 500 Hz, about 5 Hz to about 50 Hz, about 50 Hz to about 300 Hz, or about 150 Hz, and overlapping ranges therein. In some embodiments, the pulse rate may be from 1 kHz to 20 kHz. In some embodiments, a pulse width may range from, in some cases, 50 to 500 μs (micro-seconds), such as approximately 50-150,150-300, 300-500, such as 100, 200, 300, 400 μs, and overlapping ranges therein. Although frequencies below 5 kHz are used in several embodiments, some embodiments use higher frequency stimulation (e.g., of nerves at or near the wrist or ear) of 5-75 kHz (e.g., 10-40 kHz, 15-60 kHz, etc.) and a pulse width of 1-20, 10-50, 10-40 μs. The intensity of the electrical stimulation may vary from 0 mA to 500 mA, and a current may be approximately 1-11, 1-20, 5-50, 10-100 mA, and overlapping ranges therein. The electrical stimulation can be adjusted in different patients and with different methods of electrical stimulation. The increment of intensity adjustment may be, for example, 0.1 mA to 1.0 mA, such as 0.1-0.5, 0.5-0.75, 5-1 mA, and overlapping ranges therein. In some embodiments, the stimulation may last for approximately 10 minutes to 1 hour, such as approximately 10, 20, 30, 40, 50, or 60 minutes, or ranges including any two of the foregoing values. In some embodiments, stimulation may be provided for 30, 40, 50, 60, 80, 90, 120, 150 minutes 1-4 times a day. In some embodiments, stimulation occurs for 2-15 minutes (e.g., 3, 5, 7, 10 minutes) every hour (or on another interval) for a total of 40-240 minutes (e.g., 60, 80, 90, 120, 150 minutes) in a 12- or 24-hour period. Differing dosing schedules and/or differing stimulation parameters may reduce tolerance or habituation and/or may increase patient comfort/compliance. In one embodiment, beneficial effects of stimulation are provided during off periods; for example, a patient's tremor or other symptom/indication is reduced because the prior stimulation results in a prolonged effect on the nerve(s). Thus, a patient may be able to reduce the length, duration etc. of therapy over time. In some embodiments, a plurality of electrical stimuli can be delivered offset in time from each other by a predetermined fraction of multiple of a period of a measured rhythmic biological signal such as hand tremor, such as about ¼, ½, or ¾ of the period of the measured signal for example. Further possible stimulation parameters are described, for example, in U.S. Pat. No. 9,452,287 to Rosenbluth et al., U.S. Pat. No. 9,802,041 to Wong et al., PCT Pub. No. WO 2016/201366 to Wong et al., PCT Pub. No. WO 2017/132067 to Wong et al., PCT Pub. No. WO 2017/023864 to Hamner et al., PCT Pub. No. WO 2017/053847 to Hamner et al., PCT Pub. No. WO 2018/009680 to Wong et al., and PCT Pub. No. WO 2018/039458 to Rosenbluth et al., each of the foregoing of which are hereby incorporated by reference in their entireties.

In some embodiments, the electrical contacts 60 further include an optional return or ground contact for dispersing stimulation current from the body by returning to the stimulation source.

In some embodiments, the electrical contacts 60 may also provide a mechanical connection between the band 22 and the enclosure 24. In some embodiments, the electrical contacts 60 are metallic, electrically conductive snap fasteners to provide a mechanical connection.

In some embodiments, the first and/or second lugs 28, 30 may comprise protruding electrical contacts and the enclosure 24 may comprise recessed electrical contacts or some of the electrical contacts on each the first and/or second lugs 28, 30 and the enclosure 24 may be protruding and some may be recessed.

In the illustrated embedment, the electrode system 34 is mechanically coupled to the first lug 28. For example, in some embodiments, the proximal end of the electrode system 34 is sandwiched between the first lug 28 and the platform 26. The one or more lugs 28, 30 may be attached to the platform 26 by any suitable means, such as an adhesive, over molding, or permanent or removable mechanical fastener.

In certain embodiments, in addition to the engagement between corresponding shapes of the first and second lugs 28, 30 and the enclosure 24, the first and second lugs 28, 30 and/or the platform 26 can include one or more engagement structures for releasably securing the enclosure 24 to the band 22. Any suitable coupling mechanism may be employed. In the illustrated embodiment, each of the first and second lugs 28, 30 comprises one or more hooks 46 and one or more magnets 48. The one or more hooks 46 are configured to mechanical interface with a ridge or lip 66 of the enclosure 24 so as to secure the enclosure 24 to the platform 26 in some embodiments. A bottom surface of the enclosure 24 may include an aperture for the one or more hooks 46 to enter into the enclosure and secure to the ridge or lip 66. Of course, the coupling mechanism may comprise a single structure in some embodiments.

The one or more magnets 48 are configured to create a magnetic attractive force between the enclosure 24 and the band 22. In some embodiments, the enclosure 24 includes one or more magnets 48 positioned to create the magnetic attractive force with the one or more magnets 48 of the band 22 so as to secure the enclosure 24 to the platform 26. In some embodiments, the one or more magnets 48 facilitate coarse alignment of the one or more hooks 46 with the ridges or lips 66 of the enclosure 24 after which the one or more hooks 46 provide a positive lock to the enclosure 24. Other reversible connection mechanisms to connect the enclosure 24 to the band 22 can be utilized as well, including but not limited to screws, rotatable/rotational connection elements, an elastomer, and the like.

The first lug 28 can comprise an aperture 50 (e.g., a D-loop) configured to couple to a first end of the strap 36. The second lug 30 can also comprise an aperture 52 configured to receive the strap 36 so as to allow the user to adjust the length of the strap 36. The effective length of the strap 36 may be adjusted by pulling the strap 36 further through the aperture 52.

In some embodiments, the electrode system 34 comprises an aperture 58 sized and shaped to receive the strap 36. The aperture 58 can inhibit the distal end of the electrode system 34 from sliding along the limb of the user when the strap 36 is securing the band 22 to the user.

In the illustrated embodiment, the strap 36 extends from the first lug 28 on a side of the enclosure 24. In some embodiments, the strap 36 may have an adjustable length that is sufficient to accommodate any size user. In some embodiments, the strap 36 may be sized for various sizes of users (e.g., small, medium, large, child, adult, etc.). A width of the strap 36 may be less than a width of the enclosure 24 and/or the corresponding width of the platform 26.

The length (the longer dimension) of the enclosure 24 may be oriented substantially perpendicular to the length of the strap 36 and may be configured to align the length of the enclosure 24 with the length of the user's arm, leg, or other body appendage. The alignment of the length of the enclosure 24 with the length of the body part may facilitate easier movement of the body part, such as the hand and wrist, while the neurostimulation device 20 is being worn and may be generally less protrusive and awkward and, therefore, less likely to snag or inadvertently contact something in the user's environment.

In some embodiments, the strap 36 may be positioned substantially centrally along the length of the enclosure 24 and/or platform 26. In some embodiments, the strap 36 may be offset toward or near one side of the length of the enclosure 24 and/or platform 26. Offsetting the strap 36, may allow the strap 36 to be worn around, for example, the wrist of the user and the enclosure 24 to extend upward or proximally from the wrist in the direction of the shoulder rather than distally, or in the direction of the hand, which may beneficially allow or promote wrist movement (e.g., a larger range of motion).

Complementary sections of hook 54 and loop 56 fasteners (e.g., Velcro) may be attached to the strap 36 for allowing the strap 36 to form a closed loop of an adjustable length for securing the band 22 to the user, for example around the user's arm, wrist, or leg. In some implementations, the band 22 may be fabricated by attaching the sections of hook 54 and/or loop 56 fasteners to the strap 36 after the strap 36 has been received through the aperture 52.

At least a portion of the strap 36 may comprise a width that is less than a maximum width of the electrode system 34. The small width portion may be configured to be received through the aperture 58. In some embodiments, one of the complementary sections of hook 54 and loop 56 fasteners is attached to a proximal portion (e.g., adjacent to the first lug 28) and the other section 54, 56 is attached to a distal portion (e.g., at the free end of the strap 36). In some embodiments, the complementary hook and loop sections 54, 56 may be affixed on the same side of the strap 36. For example, both sections 54, 56 may be affixed to the outer surface of the strap 36, as shown in FIG. 1. The free end of the strap 36 may be wrapped over itself and the electrode system 34 to join the complementary hook and loop sections 54, 56 together. The relative positioning of the complementary hook and loop sections 54, 56 may be used to tighten or adjust the loop on the body of the user.

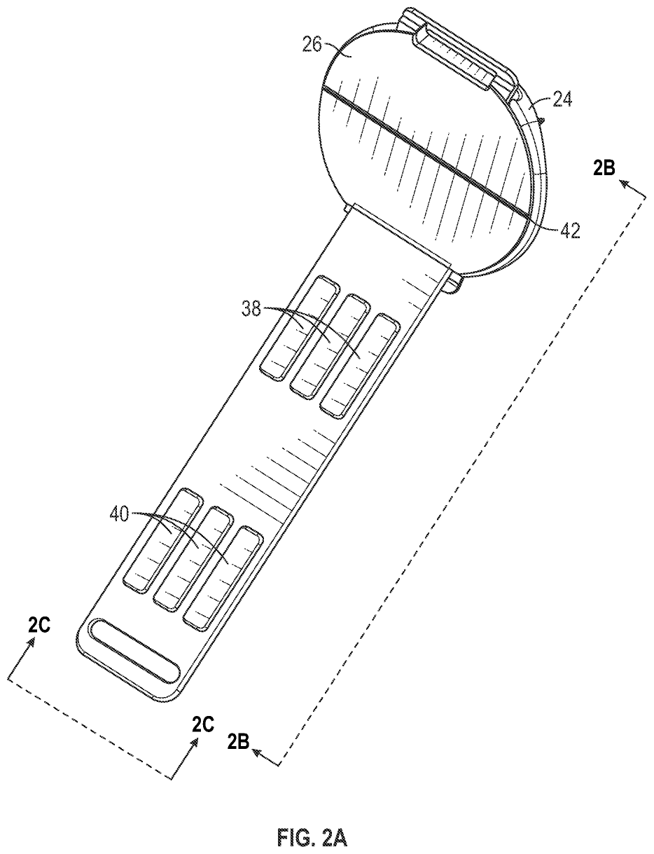

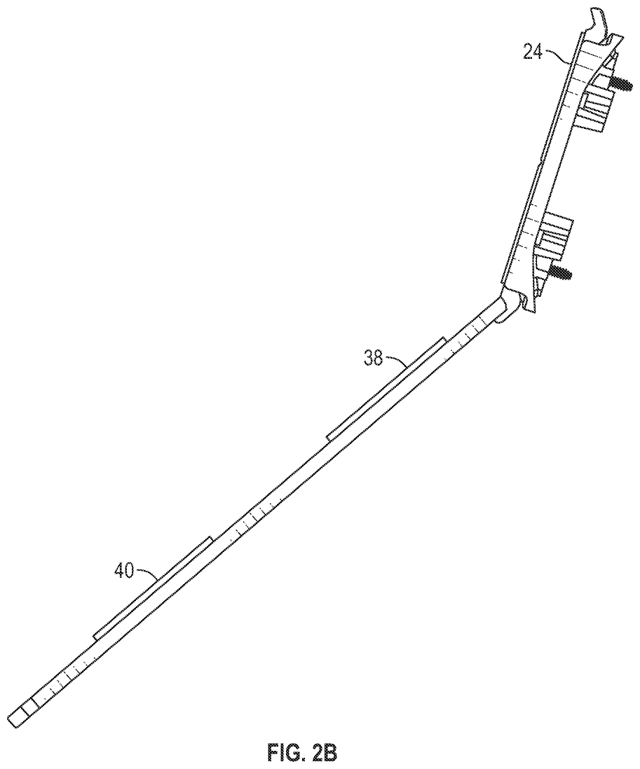

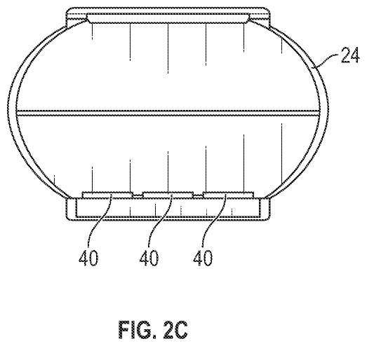

FIG. 2A is a bottom side view of the platform 26 and the electrode system 34 from FIG. 1 coupled to the enclosure 24. FIG. 2B is a side view taken along lines 2B-2B in FIG. 2A. FIG. 2C is an end view taken along lines 2C-2C in FIG. 2A. An inner side or skin side of the electrode system 34 may comprise the electrodes or electrical contacts 38, 40 configured for transcutaneously stimulating the user. FIGS. 2A-2C shows one embodiment of the size, shape, number and placement of electrodes.

In some embodiments 3-12 or more electrodes are used (e.g., 3, 6, 9 or 12). In one embodiment, none of the electrodes are in contact with regions having hair that may interfere with the stimulation or cause discomfort. For example, for wrist bands, electrodes may not be included on the back of the wrist in one embodiment. Likewise on the ankle, electrodes may be positioned in a manner that they do not contact regions with hair. For bands on the arm or leg, hair removal devices may be used. The electrodes 38, 40 could be percutaneous or microneedle electrodes in other embodiments, or only transcutaneous (e.g., not percutaneous, microneedles, or implanted electrodes in some embodiments). In many embodiments, the transcutaneous device is a wearable band or earpiece. The band may partially or fully surround a wrist, finger, arm, leg, ankle or head. Patches may be used, but in many embodiments a patch is not used.

The electrode system 34 can have any number of electrodes 38, 40 positioned between distal and proximal ends of the electrode system 34 for contacting the skin of the user. For example, 2-12 electrodes are provided (e.g., 2, 3, 4, 5, 6, 7, 8, 9, 10, 11, 12, or more). In the embodiment illustrated in FIGS. 2A-2C, the electrode system 34 has a generally rectangular shape and includes six electrodes 38, 40. In other embodiments, the electrodes 38, 40 have a round shape or any other shape. Changing the electrode shape can also control the excitation in an area and make the stimulation more comfortable.

In the illustrated embodiment, the six electrodes 38, 40 are arranged in two sets of three electrodes 38, 40 spaced along the length of the electrode system 34. Of course, the electrode system 34 is not limited to the illustrated shape or number of electrodes 38, 40. In some implementations, the electrode system 34 of the band 22 may be fabricated as a single flat piece of flexible material. Fabricating this portion as a single piece of material may simplify the manufacturing process.

As described elsewhere herein, in some embodiments, there may be one or more electrodes 38, 40 for each electrical contact 60. The electrodes 38, 40 can electrically connect to the complementary electrical contacts 60 on the enclosure 24 using electronic traces when the enclosure 24 is secured to the platform 26. The electrical contacts 60 can include stimulation contacts and/or ground contacts. In some embodiments, the electrodes may be spatially arranged in the same manner as the electrical contacts 60. In some embodiments, the electrodes 38, 40 may be arranged differently. For example, the electrodes 38, 40 may be arranged such that the electrodes 38, 40 are positioned, either axially and/or at least partially around a circumference of a body part (e.g., a wrist). In some embodiments, the electrodes 38, 40 may be configured to be generally in-line with the axon(s) of the target nerve being stimulated.

In some embodiments, the electrode system 34 employs three or more electrodes 38, 40 to apply a stimulation signal to the patient. For example, in some embodiments, at least one electrode is redundant to another electrode (e.g., 2 or more redundant common electrodes and/or 2 or more redundant stimulation electrodes). In this way, even if the electrical contact between one of the two electrodes and the patient's skin is poor increasing resistance, the electrical contact between the redundant electrode and the patient's skin can complete the electrical circuit with a normal or expected level of resistance.

In some embodiments, the 2 or more common electrodes and/or 2 or more stimulation electrodes are circumferentially spaced about the band so that even if the band rotates slightly on the wrist causing an electrode to lose contact with the patient's skin, the redundant electrode will still be in contact with the patient's skin to compete the circuit with a normal or expected level of resistance. In this way, the desired stimulation signal (e.g., frequency, phase, timing, amplitude, and/or offsets) is applied to the patient even when the band rotates on the patient's wrist. The band is less sensitive to electrical contact variations between the electrodes and the patient's skin caused by variations in the angular orientation of the band on the wrist. In some embodiments, the stimulation electrodes themselves are employed as sensing elements (e.g., for detecting electrodermal activity; or cardiac activity; or EEG) and can be placed on or proximate to a subject's wrist or placed on or proximate to a different portion of the subject's body (such as the ear, finger, portion of an arm, etc.).

As is illustrated in FIGS. 2A-2C, the platform 26 can have a groove 42 in some embodiments. In some embodiments, the platform 26 has multiple grooves 42 and/or perforations along the bend axis. In other embodiments, the platform 26 does not have the groove 42. In some embodiments, the thickness of the platform 26 varies. For example, in some embodiments, the thickness is reduced in a region between the lugs 28, 30. In the illustrated embodiment, the groove 42 is configured to reduce the bending force needed to be applied by the user to remove the enclosure 24 from the band 22. In some embodiments, when the user pinches the lugs 28, 30 together, the platform 26 takes on a bent shape, such as U-shape or V-shape, allowing the lugs 28, 30 to move slightly towards each other. In some embodiments, the platform 26 has a generally planar shape when not in the bent shape. In some embodiments, the platform 26 has a slightly bent shape even when the lugs 28, 30 are not being pinched together. In such an embodiment, the bend of the platform 26 increases when the lugs 28, 30 are pinched together. With the lugs 28, 30 moved slightly towards each other, distal ends of the hooks 46 disengage from the ridges or lips 66 allowing the user to lift the enclosure 24 off of the platform 26.

The components of the electrode system 34 can be integrated into the band 22. The advantage of this construction where the electrical contacts 60 are on the enclosure 24 is that electronics are not needed in the band 22.

FIG. 3 is a perspective view of the band 22 from FIG. 1 with one or more magnets 48 of the enclosure 24 aligned with one or more magnets 48 of the band 22 prior to attachment of the enclosure 24 to the platform 26 in part by an attractive magnetic force. In the illustrated embodiment, each of the band 22 and the enclosure 24 include two magnets 48. The disclosure is not limited to the illustrated number of magnets 48 and can include any number of magnets 48.

In some embodiments, the one or more magnets 48 on the first and second lugs 28, 30 are disposed near a center of the first and second lugs 28, 30 for connecting and/or aligning the band 22 to the enclosure 24. In other embodiments, the one or more magnets 48 are disposed in a border region of the first and second lugs 28, 30. Though the one or more magnets 48 are shown to be round, they may be of any other geometric shape.

The number and arrangement of the one or more magnets 48 may be changed in any way which facilitates attachment between the band 22 and the enclosure 24. In some embodiments, at least two magnets 48 on each of the band 22 and the enclosure 24 are desirable in order to facilitate attachment between the band 22 and the enclosure 24. In other embodiments, a single magnet 48 on each of the band 22 and the enclosure 24 may be employed. The magnets 48 may be attached to the enclosure 24 and the band 22 by various means known in the art. For example, the magnets 48 may be insert-molded or embedded into the enclosure 24 and/or the band 22 with an adhesive.

Additionally, the one or more magnets 48 may be secured within the enclosure 24 and/or the band 22 using a structure for reinforcing the magnets 48. For example, as shown in FIG. 3, the one or more magnets 48 in the band 22 are embedded within a recess 62 in each of the first and second lugs 28, 30. Similarly, as shown in FIG. 3, the one or more magnets 48 in the enclosure 24 are embedded within one or more recesses 64.

Though hooks 46 and magnets 48 are shown in the figures, in other embodiments, the hooks 46 and/or magnets 48 may be substituted with any other structure for engaging the enclosure 24 to the band 22. Non-limiting examples of such other structures include mechanical structures such as one or more holes, or recesses configured to receive protrusions, pins, Velcro, adhesives, or any combination of the above.

In some embodiments, the one or more magnets 48 may comprise a magnet or ferromagnetic material that is attracted to the magnet 48. Once the magnets 48 are close enough that they are magnetically attracted to each other, the magnetic attraction or force facilitates keeping the enclosure 24 and the band 22 together and/or in alignment. The engagement of the hooks 46 and magnets 48 may provide a physical and audible confirmation to the user that the enclosure 24 installation is complete. In some embodiments, an audible sound is heard when the enclosure 24 is engaged with the band 22.

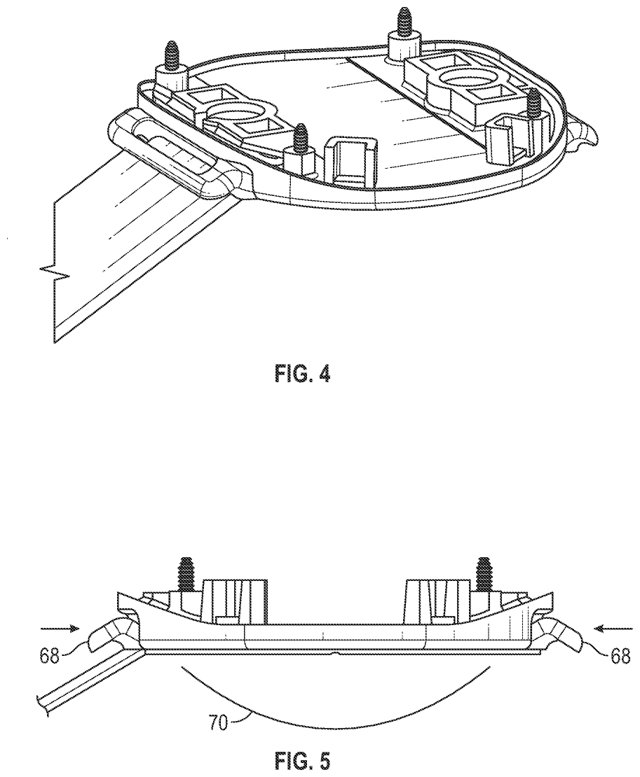

FIG. 4 is a perspective view similar to FIG. 3 except the enclosure 24 is attached to the platform 26 of the band 22. The hooks 46 are not shown in FIG. 4. FIG. 5 is a front view of the enclosure 24 and the band 22 from FIG. 4 showing opposing lugs 28, 30 that are configured to be pinched by the user to facilitate removal of the enclosure 24 from the platform 26 of the band 22. The size and shape of the first and second lugs 28, 30 may be configured to provide one or more suitable contact surfaces 68 for the user to slightly pinch the first and second lugs 28, 30 towards each other as shown in FIG. 5. A user may press one or more of his or her fingers against the contact surfaces 68 in order to advantageously bend the platform 26 into a slight U-shape or V-shape 70 allowing the hooks 46 to disengage from the edges or lips 66 in order to lift the enclosure 24 off of the platform 26. The contact surfaces 68 may allow a user to apply slight compressive pressure to the platform 26. In some embodiments, the contact surface 68 on the first lug 28 is adjacent to the aperture 50. In some embodiments, the contact surface 68 on the second lug 30 is adjacent to the aperture 52.

In some advantageous embodiments, the user is unable to sufficiently bend the platform 26 to disengage the hooks 46 from the edges or lips 66 when the band 22 is being worn by the user. For example, when the band 22 is being worn, the wrist of the user can prevent the user from sufficiently bending the platform 26 to disengage the hooks 46 from the edges or lips 66. In this way, by requiring the platform 26 to bend or flex to remove the enclosure 24, it is less likely that the enclosure 24 could inadvertently disengage from the platform 26.

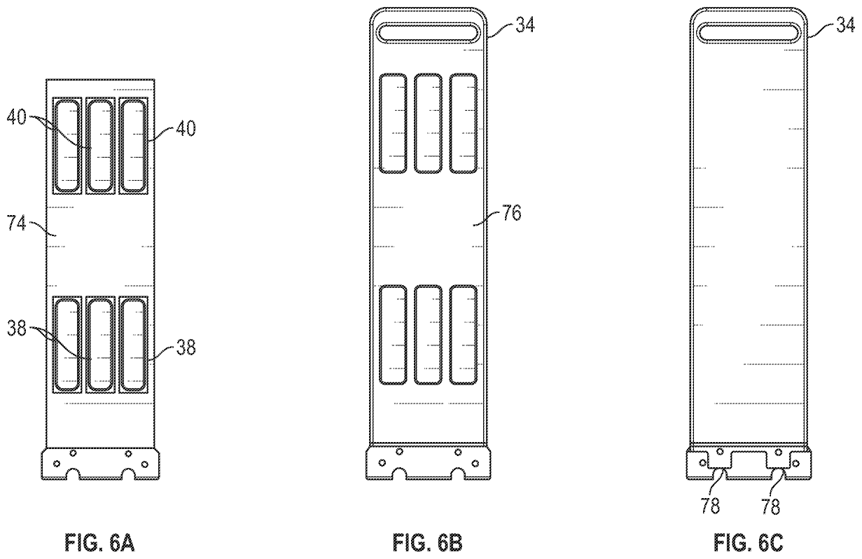

FIGS. 6A-6C disclose a manufacturing process for the electrode system 34. FIG. 6A is a top plan view of an internal layer 72 of the electrode system 34 that includes one or more electrodes 38, 40 and a conductive material 74. The one or more electrodes 38, 40 are configured to contact skin of the user. In some embodiments, the internal layer 72 is formed by over molding the one or more electrodes 38, 40 with a conductive material 74 (e.g., silicone). In this way, surfaces of the internal layer 72 expose the one or more electrodes 38, 40.

FIG. 6B is a view similar to FIG. 6A except portions of the internal layer 72 are covered by an outer layer 76 of non-conductive material leaving the one or more electrodes 38, 40 exposed to contact the skin of the user. For example, in some embodiments, the proximal end of the electrode system 34 and the one or more electrodes 38, 40 are left uncovered by the outer layer 76. FIG. 6C is a bottom plan view of the electrode system 34 from FIG. 6B. In some embodiments, the proximal end of the electrode system 34 includes one or more recesses 78 configured to engage with the first lug 28 and/or the platform 26. In some embodiments, the proximal end of the electrode system 34 includes exposed conductive surfaces for contacting the electrical contacts 60 of the enclosure 24.

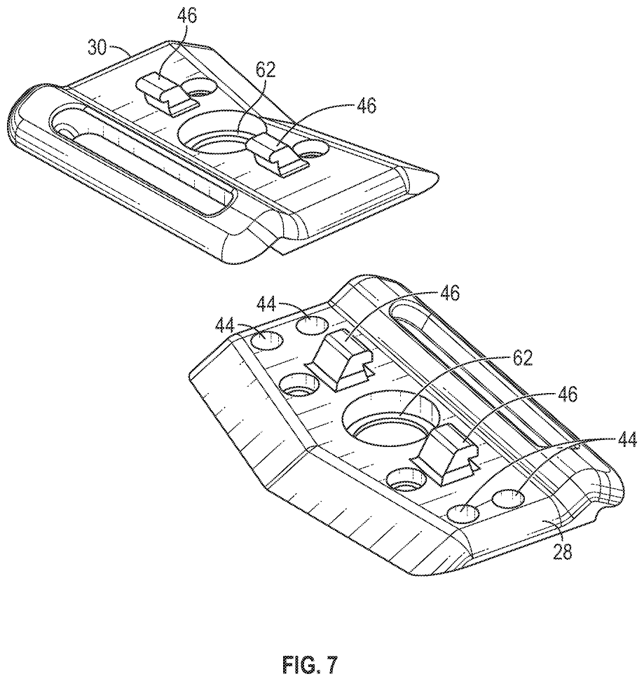

FIG. 7 is perspective views of the first lug 28 and the second lug 30 from FIG. 1. FIG. 8 is plan views of the first lug 28 and the second lug 30 from FIG. 7. In some embodiments, the first lug 28 and the second lug 30 are manufactured by molding. In some embodiments, the first lug 28 and the second lug 30 are manufactured from plastic. In some embodiments, the plastic can be any plastic such as, for example, polycarbonate (PC) and acrylonitrile butadiene styrene (ABS). In some embodiments, the first lug 28 and the second lug 30 are manufactured with recesses 62. The one or more magnets 48 can be embedded within the recesses 62 in each of the first and second lugs 28, 30. As is illustrated in FIGS. 7 and 8, the first lug 28 and the second lug 30 are molded with each including one or more engagement structures configured to secure to the enclosure 24. In the illustrated embodiment, the first and second lugs 28, 30 each include the hooks 46 and recesses 62 for the magnets 48. In the illustrated embodiment, the one or more holes 44 in the first lug 28 provide passages for the electrical contacts 60 (e.g., ground and stimulation) (see FIG. 3) from the enclosure 24 to contact the electrode system 34.

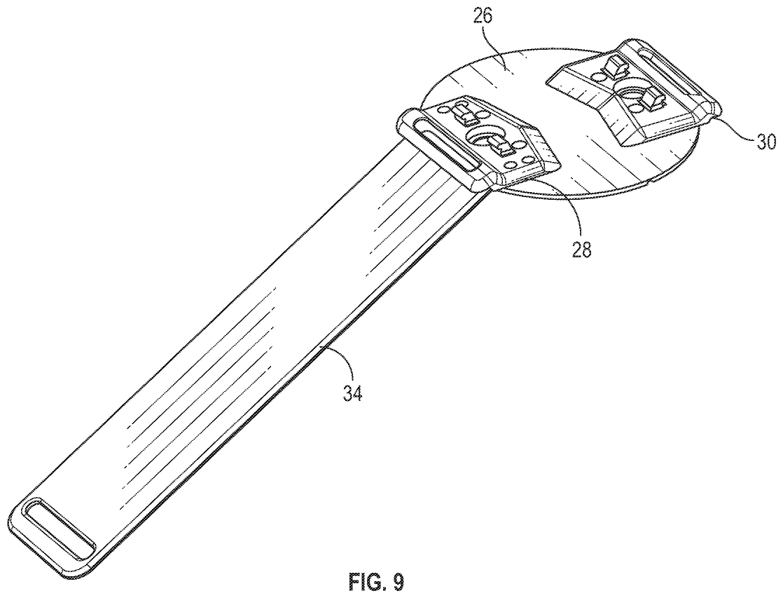

FIG. 9 is a perspective view of the electrode system 34 from FIGS. 6B-6C assembled to the platform 26 by sandwiching the proximal end of the electrode system 34 between the first lug 28 and the platform 26. The electrode system 34 is disposed so as to contact the one or more electrical contacts 60 of the enclosure 24 when the enclosure 24 is secured to the platform 26. In some embodiments, the platform 26 is over molded to the first and second lugs 28, 30 with the proximal end of the electrode system 34 sandwiched between the first lug 28 and the platform 26. In some embodiments, the one or more holes 44 in the first lug 28 align with the recesses 78 in the electrode system 34 to allow a portion of the over molded platform 26 to flow or lock to the electrode system 34 and the first lug 28. Of course, the method of manufacture is not limited to the illustrated embodiment and can be modified or changed as known to a person having ordinary skill in the art.

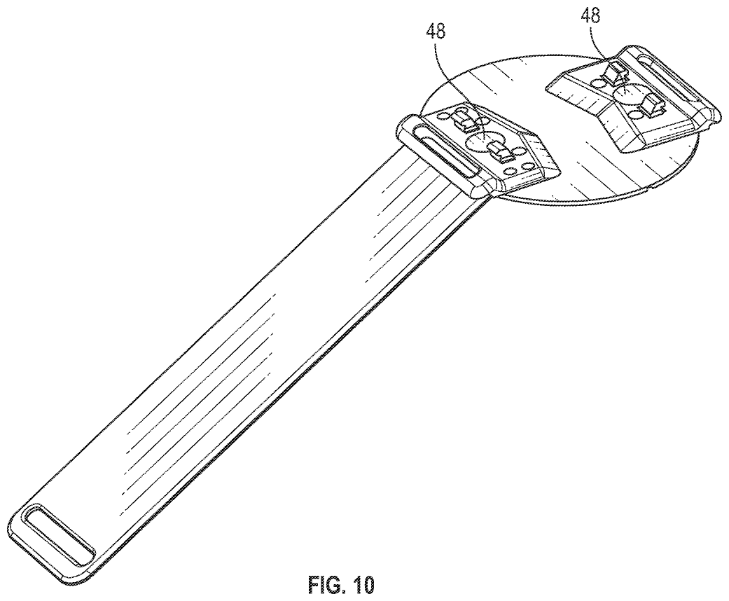

FIG. 10 is similar to FIG. 9 except the one or more magnets 48 are disposed in the recesses 62 in the first and second lugs 28, 30 to create a magnetic attraction force between the enclosure 24 and the platform 26. The device 20 can also include one, two, three, or more sensors, which can include any number of combination of inertial measurement units (IMUs) single or multi-axis accelerometers, gyroscopes, inclinometers (to measure and correct for changes in the gravity field resulting from slow changes in the device's orientation), magnetometers; fiber optic electro goniometers, optical tracking or electromagnetic tracking; electromyography (EMG) to detect firing of tremoring muscle; electroneurogram (ENG) signals; cortical recordings by techniques such as electroencephalography (EEG) or direct nerve recordings on an implant in close proximity to the nerve; heart rate or HRV sensors, galvanic skin response sensors (GSR), thermocouples, photoplethysmography sensor (PPG), temperature sensors (e.g., for body/skin temperature or ambient temperature), and/or other physiologic sensors, for example. In some embodiments, the one or more sensors can be employed to measure response to therapy as well as to calibrate therapy.

The neuromodulation devices, e.g., neurostimulation devices, described herein, in several embodiments, can be used for the treatment of depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). The devices described herein may also be used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases. The devices described herein can be used for the treatment of inflammatory skin conditions in some embodiments. The neuromodulation devices, e.g., neurostimulation devices, described herein can be used for the treatment of chronic fatigue syndrome. The devices described herein can be used for the treatment of chronic inflammatory symptoms and flare ups. Bradykinesia, dyskinesia, rigidity may also be treated according to several embodiments. In several embodiments, rehabilitation as a result of certain events is treated, for example, rehabilitation from stroke or other cardiovascular events. Systems and methods to reduce habituation and/or tolerance to stimulation in the disorders and symptoms identified herein are provided in several embodiments by, for example, introducing variability in stimulation parameter(s) described herein.

In several embodiments, the neuromodulation, e.g., neurostimulation, devices described herein can be used for the treatment of cardiac conditions (such as atrial fibrillation) and for the treatment of immune dysfunction. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s). The devices described herein can be used to stimulate the autonomic nervous system. The devices described herein can be used to balance the sympathetic/parasympathetic nervous systems. Dysfunction or imbalance of the autonomic nervous system is believed to be a potential underlying mechanism for various chronic diseases. Autonomic dysfunction develops when the nerves of the ANS are damaged or degraded. This condition is called autonomic neuropathy or dysautonomia. Autonomic dysfunction can range from mild to life-threatening and can affect part of the ANS or the entire ANS. Sometimes the conditions that cause problems are temporary and reversible. Others are chronic, or long term, and may continue to worsen over time. Examples of chronic diseases that are associated with autonomic dysfunction include, but are not limited to, diabetes, Parkinson's disease, tremor, cardiac arrhythmias including atrial fibrillation, hypertension, overactive bladder, urinary incontinence, fecal incontinence, inflammatory bowel diseases, rheumatoid arthritis, migraine, depression, and anxiety.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (including but not limited to neuroinflammation and other inflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation, etc.), headache, back pain, joint pain and stiffness, muscle pain and tension, etc. Other disorders (e.g., hypertension, dexterity, and cardiac dysrhythmias) can also be treated using the embodiments described herein.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, greater auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. The methods disclosed herein include certain actions taken by a practitioner; however, they can also include any third-party instruction of those actions, either expressly or by implication. For example, actions such as “percutaneously stimulating an afferent peripheral nerve” includes “instructing the stimulation of an afferent peripheral nerve.”

What is claimed is:

1. A band for securing an enclosure of a wearable neurostimulation device for modulating one or more peripheral nerves of a user, the band comprising:
a flexible platform configured to bend from a first shape to a second shape different than the first shape responsive to an applied force;
a plurality of lugs supported by the flexible platform, the plurality of lugs having engagement structures configured to mechanically engage with the enclosure of the wearable neurostimulation device when the flexible platform is in the first shape and disengage from the enclosure when the flexible platform is moved to the second shape responsive to the applied force; and
an electrode system coupled to the flexible platform and having an inner side and an outer side, the inner side comprising at least one electrode for each nerve to be stimulated.

2. The band of claim 1, wherein the first shape is generally planar and the second shape is curved.

3. The band of claim 1, wherein the engagement structure comprises one or more hooks.

4. The band of claim 1, further comprising one or more magnets, the one or more magnets being configured to create a magnetic attractive force with the enclosure when the enclosure is in close proximity to the band.

5. The band of claim 1, wherein the flexible platform comprises a groove configured to reduce a bending force required to bend the flexible platform from the first shape to the second shape.

6. The band of claim 1, wherein at least one of the plurality of lugs comprises one or more holes for providing a passage for an electrical contact from the enclosure to contact the electrode system.

7. The band of claim 1, wherein the electrode system comprises a distal end and a proximal end, and wherein the electrical contact from the enclosure is configured to contact the distal end.

8. A band for securing an enclosure of a wearable neurostimulation device that stimulates one or more peripheral nerves of a user, the band comprising:
a platform configured to bend from a first shape to a second shape different than the first shape;
an engagement structure configured to secure the enclosure to the platform when the platform is in the first shape and disengage from the enclosure when the platform is in the second shape; and
a plurality of contact surfaces configured for the user to bend the platform from the first shape to the second shape.

9. The band of claim 8, further comprising an electrode system coupled to the flexible platform and having an inner side and an outer side, the inner side comprising at least one electrode for each nerve to be stimulated.

10. The band of claim 8, wherein the first shape is generally planar and the second shape is curved.

11. The band of claim 8, wherein the engagement structure comprises on or more hooks.

12. The band of claim 8, further comprising one or more magnets, the one or more magnets being configured to create a magnetic attractive force with the enclosure when the enclosure is in close proximity to the band.

13. The band of claim 8, wherein the platform comprises a groove configured to reduce a bending force required to bend the platform from the first shape to the second shape.

14. A band for securing an enclosure of a wearable neurostimulation device that stimulates one or more peripheral nerves of a user, the band comprising:
a platform configured to support the wearable neurostimulation device;
an electrode system coupled to the platform and having an inner side and an outer side, the inner side comprising at least one electrode for each nerve to be stimulated; and
a strap coupled to the platform and being sized and shaped to wrap around the outer side of the electrode system when encircling a limb of the user.

15. The band of claim 14, wherein the electrode system comprises a distal end and a proximal end, and wherein only the proximal end is coupled to the platform.

16. The band of claim 14, wherein the electrode system comprises one or more electrical traces extending between the proximal end and the at least one electrode.

17. The band of claim 14, wherein the coupling between the electrode system and the platform includes a mechanical coupling and an electrical coupling.

18. The band of claim 14, wherein the strap is configured to be tightened against the outer surface of the electrode system forcing the at least one electrode firmly against skin of the user.

19. The band of claim 14, wherein the distal end of the strap comprises an aperture sized and shaped to slidingly receive the strap.

20. The band of claim 19, wherein the aperture is configured to inhibit the distal end of the electrode system from sliding along the limb of the user when both ends of the strap are secured relative to the platform.
